(19) United States
(12) Patent Application Publication
(10) Pub. No.: US 2010/0267996 A1
(43) Pub. Date: Oct. 21, 2010

(54) Alcohol Dehydration

(76) Inventors: Duncan Frank Wass, Bristol (GB); Igor Viacheslavovich Shishkov, Mannheim (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/808,592

(22) PCT Filed: Dec. 19, 2008
(86) PCT No.: PCT/GB08/51215

§ 371 Date: Jun. 16, 2010

(30) Foreign Application Priority Data
Dec. 20, 2007 (GB) 0724915.4
Jun. 9, 2008 (GB) 0810499.4

(51) Int. Cl. C07C 41/09; C07C 1/24; B01J 31/22; B01J 31/18

(52) U.S. Cl. 568/698; 585/639; 502/156; 502/169; 502/171; 502/167; 502/162

(57) ABSTRACT

Catalyst compositions are disclosed exhibiting activity for dehydrating an alcohol, the composition comprising a source of a Group VIII transition metal, an organic salt, an acid and/or a compound consisting of a conjugate base of an acid bonded to a radical of the alcohol to be dehydrated and, optionally, a ligand. Also disclosed are methods of converting an alcohol into a product using the catalyst composition. The product of the methods may be predominately alkene or ether depending on the method. In some embodiments of the method a second catalyst for converting a product into a further product may be present.

The present invention relates to catalyst compositions and to their use in the treatment of alcohols to effect dehydration and/or other reactions.

The invention may be of utility in reducing dependence on fossil fuels, both as energy sources and as sources of chemical raw materials.

The current structure of the petrochemical industry relies on the conversion of crude oil to a small number of simple building blocks (CO, ethylene, aromatics) which are then further transformed to higher value end products. There are good reasons for this approach, developed over decades of operation, including economies of scale and integration. Chemical producers are increasingly looking for alternative biosustainable routes to existing commodity products. What are needed are ways to transform simple biomass-derived building blocks, and bioethanol is an excellent example, into more complex products.

A key step in the utilisation of bioethanol is its conversion to ethylene, already used in huge volume as an intermediate in the chemical industry. This ethylene can then be easily converted to end products. The reaction required is alcohol dehydration:

Ethanol(C₂H₅OH)→Ethylene(C₂H₄)+H₂O

This reaction is known to proceed in the presence of a range of acid catalysts. However, such catalysts only operate at high temperature (>150° C. and typically 200-300° C.) and are not very selective, giving large quantities of the side-product diethyl ether unless even higher temperatures are used.

It is an aim of the present invention to provide catalysts that operate at lower temperatures. This would both lead to energy savings and allow such catalysts to be used in a so-called ‘tandem catalysis’ concept with other types of catalysts that are not thermally robust. Of course the utility of our catalyst systems is not restricted to the use of ethanol (“bio” or otherwise) as starting material.

On other occasions, there may be a need to selectively dehydrate alcohols to produce ethers; for example, bioethanol can be dehydrated to diethyl ether which finds application as a solvent, or higher alcohols such as biobutanol can be dehydrated to longer chain ethers which may be used as fuels or fuel additives. Again, there is a need for catalysts which are selective for this transformation at low temperatures.

In a first aspect the invention provides a catalyst composition capable of catalysing the dehydration of alcohols. Some preferred embodiments are surprisingly active for the dehydration of alcohols to alkenes, even at temperatures less than 150° C. (although their use is not limited to temperatures less than 150° C.). Other preferred embodiments are surprisingly active for the dehydration of alcohols to ethers; even at temperatures lower than 150° C. Preferred embodiments show good selectivity, either in terms of a high, or a low ratio of alkene to ether in the products.

In a second aspect the invention provides a method of using a catalyst according to the first aspect to effect the conversion of an alcohol into a product in a method comprising a step of dehydrating an alcohol.

In further aspects the invention provides “tandem catalysis” compositions including catalyst compositions according to the first aspect, and further including components providing the ability to catalyse further reaction(s); and methods of tandem catalysis employing such compositions.

In further aspects the invention provides catalyst compositions according to the first aspect which in certain embodiments and/or under certain conditions produce ethers as the main products, i.e. a good selectivity to ether in terms of alkene to ether ratio in the products; and methods of ether formation employing such compositions.

The catalyst composition of the first aspect comprises:

(1) A source of a Group VIII transition metal (i.e. one or more of Fe, Ru, Os, Co, Rh, Ir, Ni, Pd and Pt, preferably selected from Co, Rh and Ir, more preferably Rh or Ir);
(2) An organic salt;
(3) An acid and/or a compound as produced by reaction of an acid and said alcohol which is to be dehydrated, said compound generally consisting of a conjugate base of an acid bonded to a radical of said alcohol (typically a compound of the form RB where R is a radical derived from an alcohol ROH, and B is a conjugate base of an acid HB); and, optionally
(4) A ligand.

(1) The source of a group VIII metal can be the metal itself (which may be dispersed on a support material such as carbon, silica or alumina); or a compound of the metal, preferably a complex comprising a species of formula

[M(L)ₙ]ₘ

(which may be charged) where M is a Group VIII metal, preferably Co, Rh or Ir, more preferably Rh.

The L groups may be the same or different and are ligands, for example halide (e.g. chloride, bromide, iodide), hydride, alkoxide, amide, acetate, acetylacetonate, amine, ether, water, CO, NO, phosphines (e.g. triphenylphosphine, trimethyl phosphine, trimesityl phosphine or triphenoxyphosphine), pyridine, alcohols, alkenes, alkynes, or N-heterocyclic carbenes. L groups may also be solid state materials that act as ligands and produce a supported metal species, for example silica, alumina, zeolites or poly(vinyl pyridine).

n is an integer from 1 to 8, preferably from 2 to 6.

m is an integer representing the nuclearity of the complex (so when m=1 the complex is a monomer, when m=2 the complex is a dimer, etc.) and is generally from 1 to 8, preferably 1 or 2. Generally the nature and number of the ligands L is selected to achieve suitable stability of the complex. A single source of a Group VIII transition metal or a mixture of two or more sources may be used. It is preferred that a single source is used.

(2) The organic salt is a compound of formula

Z(X)ₚ

where Z is a cationic organic fragment; preferably a fragment of formula

[Y(R¹)₄]⁺

where Y is N, P or As, preferably N or P, more preferably N. R¹ groups can be the same or different and are H, hydrocarbon groups or heteroatom-substituted hydrocarbon groups. Suitable hydrocarbon groups are linear, branched or cyclic alkyl groups with 1 to 50 carbon atoms, (for example: methyl, ethyl, n-propyl, i-propyl, butyl, pentyl, hexyl, octyl, decyl, cyclopentyl, cyclohexyl); or aryl or substituted aryl groups (for example phenyl, ortho-tolyl, meta-tolyl, para-tolyl, ethylphenyl, isopropylphenyl, t-butylphenyl, 2,6-dimethylphenyl, 2,4-dimethylphenyl, 3,5-dimethylphenyl, 2,6-diisopropylphenyl, 2,4,6-trimethylphenyl, 2,4,6-triisopropylphenyl, naphthyl, benzyl). Suitable heteroatom-substituted hydrocarbon groups may have one or more heteroatoms (for example CF₃, CF₂CF₃, CH₂OMe, CH₂NMe₂, CH₂CH₂NH₂, CH₂CH₂N(R⁵)₂, CH₂CH₂P(R³)₂, CH₂CH₂CH₂P(R⁵)₂, fluorophenyl, perfluorophenyl, chlorophenyl, bromophenyl, C₆H₄(CF₃), C₆H₃(CF₃)₂, C₆H₄(OMe), C₆H₃(OMe)₂, C₆H₄(N(R⁵)₂), C₆H₄(P(R⁵)₂), where R⁵ is selected from H, hydrocarbon groups or heteroatom-substituted hydrocarbon groups. Suitable hydrocarbon groups are linear, branched or cyclic alkyl groups with 1 to 50 carbon atoms, (for example: methyl, ethyl, n-propyl, i-propyl, butyl, pentyl, hexyl, octyl, decyl, cyclopentyl, cyclohexyl); or aryl or substituted aryl groups (for example phenyl, ortho-tolyl, meta-tolyl, para-tolyl, ethylphenyl, fluorophenyl, perfluorophenyl, chlorophenyl, bromophenyl, C₆H₄(CF₃), C₆H₃(CF₃)₂, C₆H₄(OMe), C₆H₃(OMe)₂, isopropylphenyl, t-butylphenyl, 2,6-dimethylphenyl, 2,4-dimethylphenyl, 3,5-dimethylphenyl, 2,6-diisopropylphenyl, 2,4,6-trimethylphenyl, 2,4,6-triisopropylphenyl, naphthyl, benzyl). Suitable heteroatom-substituted hydrocarbon groups may have one or more heteroatoms (for example CF₃, CF₂CF₃, CH₂OMe, CH₂NMe₂, CH₂CH₂NH₂. When there are plural R⁵ groups, they may be the same or different. Two or more R¹ groups may also be linked so as to form a cyclic structure.

Alternatively, Z may be a heterocycle of formula

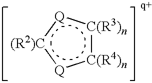

The broken line within the ring structure merely indicates that one or more pairs of adjacent ring atoms are multiply bonded.

The Q groups may be the same or different and are O, S, N(R¹), P(R¹) or C(R¹)₂ where R¹ is as defined above; preferably Q groups are the same and are N(R¹).

The groups R²—R⁴ may be the same or different and have the same definition as R¹.

The values of n may be the same or different and are each 1 or 2.

When n is 1, the carbon atom to which the R³ or R⁴ group is attached is sp² hybridised. It may be connected to the adjacent Q group or the adjacent C by a double bond.

Any of the groups R¹-R⁴ may be linked to form cyclic structures.

The amount of the charge, q, is a small integer, usually 1.

X is an anion; for example F, Cl, Br, I, acetate, triflate, tosylate, BF₄, AlCl₄, PF₆, ClO₄, BPh₄, B(C₆F₅)₄, B[3,5-(CF₃)₂C₆H₄]₄, or Al(OC₄F₉)₄.

The value of p is such as to balance the charge of the overall salt; for example, if Z is a dication and X is a monoanion, p=2; or, if Z is a monocation and X is a dianion, p=0.5. In most cases, and preferably, Z will be a monocation and X a monoanion and p=1.

A single organic salt or a mixture of two or more salts may be used. It is preferred that a single salt or a mixture of two salts is used.

(3) The acid may be a Brønsted acid or a Lewis acid, preferably a Brøonsted acid. It will generally be a relatively strong acid, such that it could by itself catalyse the dehydration of alcohols, albeit at high temperatures. Examples of suitable acids are HF, HCl, HBr, HI, tosylic acid, triflic acid and other fluorinated organic acids, HBF₄, acetic acid, solid-state acids (such as certain zeolites, aluminas, clays and silicas), and heteropolyacids (such as H₃PW₁₂0₄₀) It is preferred that the acid is HBr or HI, more preferably HI. The role of the acid is to react with the alcohol substrate (ROH) to produce a compound of formula RB where B is the conjugate base of the acid used. The acid may therefore be pre-reacted with some of the alcohol substrate to produce RB before addition of the other catalyst components. In this way pre-formed RB may be used as an alternative to the acid in the overall catalyst composition.

A single acid or a mixture of two or more acids may be used. It is preferred that a single acid is used.

(4) The optional ligand may be a monodentate or polydentate C—, N—, P, As-, O- or S-donor ligand. In the case of polydentate ligands, the ligand may contain the same or different types of donor. It is preferred that the optional ligand is based on C-donors (for example CO or carbenes, such as N-heterocyclic carbenes), N-donors (for example, amine, pyridine, bipyridine), O-donors (for example acetate or acetyl acetonate) or P-donors (for example; phosphines, such as triphenylphosphine, tritolylphosphine, trimesitylphosphine, trimethylphosphine, triethylphosphine, tricyclohexylphosphine, diphenylphosphinochloride, phenylphosphinodichloride, 1,2-bisdiphenylphosphinoethane, 1,3-bisdiphenylphosphinopropane, or triphenoxyphosphine). The ligand may enhance the stability of the composition.

A single ligand or a mixture of two or more ligands may be used. It is preferred that a single ligand or a mixture of no more than four ligands is used; it is more preferred that a single ligand or a mixture two ligands is used.

Irrespective of the precise composition of the catalyst system, the various components of the catalyst may be present in a range of ratios.

Typically, the ratio of the moles of the Group VIII transition metal in component (1) to the organic salt (2) will be in the range 1:1 to 1:10⁸, preferably 1:10 to 1:10⁶ and more preferably 1:100 to 1:10⁵. Typically, the ratio of the moles of the Group VIII transition metal in component (1) to the acid (3) will be in the range 1:1 to 1:10⁸, preferably 1:10 to 1:10⁶ and more preferably 1:100 to 1:10⁵. Typically, the ratio of the moles of the Group VIII transition metal in component (1) to the optional ligand (4) will be in the range 0.01:1 to 1:100, preferably 0.1:1 to 1:10 and more preferably 1:1 to 1:5.

The catalytic components may be pre-mixed in any order before a catalytic dehydration reaction or added in situ, in the presence of the alcohol to be dehydrated.

The catalytic dehydration reaction process may be run continuously or as a batch reaction, in a wide variety of reactor configurations known to those skilled in the art.

A very wide range of alcohols may be dehydrated by this process; for example ethanol; other hydrocarbyl alcohols, such as n-propanol, i-propanol, butanol, pentanol, hexanol, octanol or longer chain alcohols; diols such as butanediol; alcohols with other organic functionality, such as ether, ester, carboxylic acid, aromatic groups, or amines. The preferred alcohols are ethanol, butanol and hexanol and other hydrocarbyl alcohols. The alcohol may be in the liquid or gas phase depending on the precise nature of the alcohol and the reaction temperature.

The reaction conditions and catalyst composition may be chosen so that the product of the method is predominantly alkene or predominantly ether. Thus, the selectivity of the method may be such that the alkene:ether ratio of the products (for the case where the product is predominantly alkene) is 10⁴:1 to 1.1:1, preferably 500:1 to 1.1:1, more preferably 50:1 to 1.1:1. Alternatively, the selectivity of the method may be such that the alkene:ether. ratio of the products (for the case where the product is predominantly ether) is 1:10⁴ to 1:1.1, preferably 1:500 to 1:1.1, more preferably 1:1.1 to 1:200. These ratios are preferably molar ratios.

Preferably, the method is so selective for ether that the alkene products are at too low a concentration to be detected. Alternatively, the method is preferably so selective for alkene that the ether products are at too low a concentration to be measured.

The reaction temperature may range from 0° C. to 500° C., preferably from 0° C. to 300° C., more preferably from 50° C. to 200° C., and even more preferably from 70° C. to 150° C. The reaction may optionally be run in a diluent, such as an organic solvent (for example; alkanes such as pentane, hexane, heptane; aromatic compounds such as benzene, toluene, xylenes; alcohols such as ethanol, propanol, hexanol; and ethers such as diethyl ether, dyglyme, water, super critical CO₂, an ionic liquid (such as the organic salt component of the catalyst system) or a mixture of two or more of these diluents. In some cases, no additional diluent is used so the reaction is carried out with the alcohol substrate, water and dehydration products acting as diluents.

### Tandem Catalysis

The catalyst system for the dehydration of alcohols to alkenes and/or ethers may be used alone, or in conjunction with a second catalyst in the same reactor. This second catalyst may be any catalyst capable of converting the alkene and/or ether produced in the dehydration reaction into a further product preferably under the conditions employed for the dehydration reaction. For example, if ethanol is used as the alcohol substrate, the method is selective for alkenes and the second catalyst is an ethylene dimerisation catalyst, the overall reaction observed will be the conversion of ethanol to butenes (ethanol dehydrated to ethylene: ethylene dimerised to butenes). In some cases, the product of tandem catalysis may be further transformed under the reaction conditions to another product; for example, butenes may be rehydrated to butanols. Examples of suitable second catalysts include catalysts for the dimerisation (such as nickel and palladium phosphine catalysts), trimerisation (such as chromium catalysts), oligomerisation (such as nickel, cobalt or iron catalysts) or polymerisation (such as late transition metal catalysts) of alkenes. A wide variety of such catalysts are known for all of these processes and may reasonable be applied in this application. For example, second catalysts based on any one of the compounds 11, 18, 19, 20, 21 and/or 22 discussed in the Examples may be used. Generally, second catalysts will comprise a source of a Group VIII metal (e.g. Fe, Ru, Os, Co, Rh, Ir, Ni, Pd, Pt) preferably Fe, Ru, Fe, Ni, Pd or Pt.

In some cases, the catalyst system for the dehydration of alcohols to alkenes may itself also be a catalyst for the dimerisation, oligomerisation or polymerisation of the alkene, so that the second catalyst in a tandem catalyst reaction may have the same composition as the dehydration catalyst, i.e. a single suitable catalyst composition may act both as the dehydration catalyst and as the second catalyst.

Preferably the dehydration reaction uses conditions and a catalyst composition so that the product is predominantly alkene.

### Dehydration to Ethers

The catalyst system for the dehydration of alcohols may in some embodiments and under certain reaction conditions produce the ethers which are usually considered side-products for alkene formation in such quantity that good selectivity to ethers rather than alkenes is achieved. In these cases the selective production of ethers or the co-production of alkenes and ethers may be the desired outcome, and in general catalyst embodiments and reaction conditions which are less preferred for selective alkene production will be more preferred for ether production.

A single alcohol may be dehydrated in this way to an ether in which both alkyl groups are the same; for example, ethanol can be dehydrated to diethyl ether, or butanol to dibutyl ether. Alternatively, a mixture of alcohols may be dehydrated to an ether in which the alkyl groups are different; for example, a mixture of ethanol and butanol may be dehydrated to butyl ethyl ether.

### EXAMPLES

### General Considerations

All procedures were carried out under an inert (N₂) atmosphere using standard Schlenk line techniques or in an inert atmosphere (Ar) glovebox. Chemicals were obtained from Sigma Aldrich, Strem or Fisher Scientific and used without further purification unless otherwise stated. Reaction products were analysed by GC-FID (Hewlett Packard Series) using an Alltech Econo-CAP column, ECS, 30 m×0.25 mm, IDX 0.25 μm. All percentages given are based on mole percentages vs the substrate alcohol.

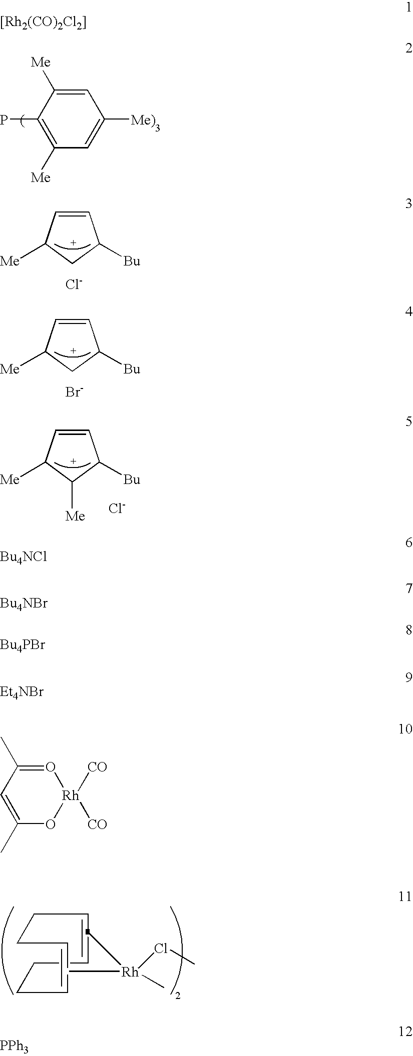

### Examples 1-14

### See Table 1

Hexan-1-ol (3.0 ml, 24 mmol) was added to a round bottom flask containing the required amounts of Group VIII transition metal compound, organic salt and (when used) ligand. HI (1.5 mmol as 57% solution in water) was added and the mixture was heated to the required reaction temperature. After 48 hours, the reaction mixture was allowed to cool to room temperature and reaction products were analysed by GC-FID.

TABLE 1

| | Compound | | Ligand | | Salt | Temp | Hexenes | Hexyl ether |
|---|---|---|---|---|---|---|---|---|
| Example | (%) | | (%) | | (%) | (° C.) | (%) | (%) |
| 1 | 1 | (0.065) | 2 | (0.13) | 3 (14.4) | 110 | 24.8 | 1.9 |
| 2 | 1 | (0.065) | none | | 3 (14.4) | 110 | 15.2 | 1.2 |
| 3 | 1 | (0.065) | 2 | (0.13) | 4 (14.4) | 110 | 36.9 | 9.7 |
| 4 | 1 | (0.065) | 2 | (0.13) | 5 (14.4) | 110 | 17.0 | 1.0 |
| 5 | 1 | (0.065) | 2 | (0.13) | 6 (14.4) | 110 | 16.9 | 0.7 |
| 6 | 1 | (0.065) | 2 | (0.13) | 7 (14.4) | 110 | 46.2 | 18.8 |
| 7 | 1 | (0.065) | 2 | (0.13) | 8 (14.4) | 110 | 21.8 | 15.1 |
| 8 | 1 | (0.065) | 2 | (0.13) | 9 (14.4) | 110 | 13.3 | 1.0 |
| 9 | 10 | (0.5) | none | | 7 (21.6) | 110 | 80.2 | 16.9 |
| 10 | 11 | (0.5) | none | | 7 (21.6) | 110 | 48.5 | 48.1 |
| 11 | 1 | (0.26) | 2 | (0.52) | 7 (21.6) | 110 | 86.9 | 13.0 |
| 12 | 1 | (0.065) | 12 | (0.13) | 3 (14.4) | 110 | 3.0 | 0.1 |
| 13 | 1 | (0.065) | 2 | (0.13) | 4 (14.4) | 100 | 33.1 | 8.2 |
| 14 | 1 | (0.065) | 2 | (0.13) | 4 (14.4) | 90 | 16.6 | 2.2 |

### Examples 15-23

### See Table 2

The same method was followed as described for examples 1-14, only the reaction was performed in a 300 ml capacity stainless steel autoclave at 110° C. and 4 ml of the required alcohol as indicated was used instead of hexan-1-ol.

Compounds 13-15 were synthesised by reaction of 1 with Et₄NCl, Et₄NBr or Et₄NI respectively. Compound 16 was synthesised by reaction of 1 with excess I₂.

### 13 [Et₄N][Rh(CO)₂Cl₂]

### 14 [Et₄N][Rh(CO)₂Br₂]

### 15 [Et₄N][Rh(CO)₂I₂]

### 16 [Et₄N]₂[Rh(CO)I₅]

### 17 Et₄NI

TABLE 2

| | | Compound | | Ligand | Salt | | alkene | ether |
|---|---|---|---|---|---|---|---|---|
| Example | Alcohol | (%) | | (%) | (%) | | (%) | (%) |
| 15 | Pentan-1-ol | 1 | (0.065) | 2 (0.13) | 7 | (14.4) | 26.0 | 17.9 |
| 16 | Butan-1-ol | 1 | (0.065) | 2 (0.13) | 7 | (14.4) | 94.0 | 1.0 |
| 17 | Propanol | 1 | (0.065) | 2 (0.13) | 7 | (14.4) | 90.8 | 5.0 |
| 18 | Ethanol | 1 | (0.065) | 2 (0.13) | 7 | (14.4) | 44.0 | 39.0 |
| 19 | Ethanol | 13 | (0.065) | none | 17 | (14.4) | 40.0 | 2.0 |
| 20 | Ethanol | 14 | (0.065) | none | 17 | (14.4) | 45.0 | 6.0 |
| 21 | Ethanol | 15 | (0.065) | none | 17 | (14.4) | 50.0 | 10.0 |
| 22 | Ethanol | 16 | (0.065) | none | 17 | (14.4) | 15.0 | 2.0 |
| 23 | Ethanol | 15 | (0.065) | none | 17 | (0.13) | 35.0 | 8.0 |

### Comparative Example 1

An identical experiment to example 1 was performed only no Group VIII transition metal compound or organic salt was added. The reaction product was 2.5% hexyl ether: no hexenes were detected. This comparative example demonstrates that HI alone will not dehydrate alcohols to form the desired alkenes at this temperature.

### Comparative Example 2

An identical experiment to example 1 was performed only no Group VIII transition metal compound was added. The only reaction product detected was dihexyl ether. This comparative example demonstrates a Group VIII transition metal compound is required to dehydrate alcohols to form the desired alkenes.

### Comparative Example 3

An identical experiment to example 1 was performed only no organic salt was added. The reaction product was 1.1% hexyl ether: no hexenes were detected. This comparative example demonstrates an organic salt is required to dehydrate alcohols to form the desired alkenes, and that performance in its absence is poor.

### Comparative Example 4

An identical experiment to example 1 was performed only no acid was added. No reaction products were detected. This comparative example demonstrates an acid is required to dehydrate alcohols to form the desired alkenes.

### Examples 24-29

### (Table 3): Tandem Catalysis

The same method as example 18 was performed only 0.1 mmol of a second catalyst was introduced. If the second catalyst is omitted, as in example 18, less than 2% of butenes are observed showing that the second catalyst is acting to significantly increase the amount of dimerisation of the ethene produced.

Compounds 18, 19, 20, 21 or 22 may be synthesised by those skilled in the art by reaction of a suitable palladium or platinum precursor compound, for example [Pd(1,5-cyclooctadiene)MeCl], and the appropriate ligand. The ligand in Compound 20 was synthesised according to the procedure described in J. Org. Chem. 2002, vol. 67, pages 443-449 and the ligand in compounds 21 and 22 was synthesised according to the procedure described in Inorg. Chem. 1989, vol. 28, pages 1624-1627.

TABLE 3

| | |
|---|---|
| 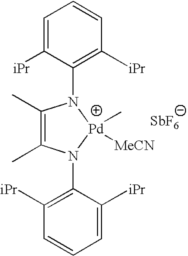 18 | 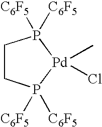 19 |

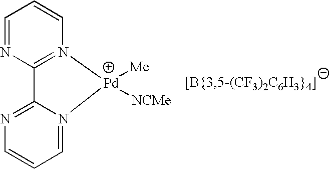

20 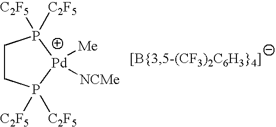
21 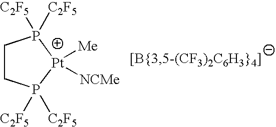
22

| Example | Second catalyst | Butenes (%) |
|---|---|---|
| 24 | 11 | 3.5 |
| 25 | 18 | 4 |
| 26 | 19 | 3.5 |
| 27 | 20 | 6 |
| 28 | 21 | 10 |
| 29 | 22 | 7 |

### Examples 30-33

### (See Table 4): Dehydration to Ethers

Hexan-1-ol (3.0 ml, 24 mmol) was added to a round bottom flask containing the required amounts of Group VIII transition metal compound, organic salt and (when used) ligand. HI (1.5 mmol as 57% solution in water) was added and the mixture was heated to the required reaction temperature. After 48 hours, the reaction mixture was allowed to cool to room temperature and reaction products were analysed by GC-FID.

TABLE 4

| | | |
|---|---|---|
| [RhCl₃] 23 | [IrCl₃] 24 | 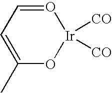 25 |

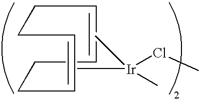

26

| Ex- | Com- | | | | | |
|---|---|---|---|---|---|---|
| am- | pound | Ligand | Salt | Temp | Hexenes | Hexyl |
| ple | (%) | (%) | (%) | (° C.) | (%) | ether (%) |
| 30 | 23 | none | 7 | 110 | 0 | 18 |
| | (0.065) | | (21.6) | | | |
| 31 | 24 | none | 7 | 110 | 0 | 31 |
| | (0.065) | | (21.6) | | | |
| 32 | 25 | none | 7 | 110 | 0 | 35 |
| | (0.065) | | (21.6) | | | |
| 33 | 26 | none | 7 | 110 | 0 | 33 |
| | (0.065) | | (21.6) | | | |

1. A catalyst composition exhibiting activity for dehydrating an alcohol, the composition comprising
i) a source of a Group VIII transition metal;
ii) an organic salt;
iii) an acid and/or a compound consisting of a conjugate base of an acid bonded to a radical of the alcohol to be dehydrated; and, optionally
iv) a ligand.

2. A method of converting an alcohol into a product comprising a step of dehydrating the alcohol by means of a catalyst composition which comprises:
(i) a source of a Group VIII transition metal;
(ii) an organic salt;
(iii) an acid and/or a compound consisting of a conjugate base of an acid bonded to a radical of said alcohol which is to be dehydrated; and, optionally
(iv) a ligand.

3. A method according to claim 2 in which the metal is at least one of Co, Rh and Ir.

4. A method according to claim 3 in which the metal is Rh or Ir.

5. A method according to any preceding claim in which the metal source is in the form of elemental metal, optionally dispersed on a support material.

6. A method according to any of claims 1-3 in which the metal source is a complex comprising a species of formula
[M(L)ₙ]ₘ
(which may be charged) where M is the Group VIII metal;
the L groups, which may be the same or different, are ligands;
n is an integer from 1 to 8; and
m is an integer representing the nuclearity of the complex.

7. A method according to claim 6 in which the ligands L are selected from chloride, bromide, iodide, hydride, alkoxide, amide, acetate, acetylacetonate, amine, ether, water, CO, NO, phosphines, pyridine, alcohols, alkenes, alkynes, N-heterocyclic carbenes, and solid state materials that act as ligands and produce a supported metal species.

8. A method according to any preceding claim wherein the organic salt is a compound of formula
Z(X)ₚ
where Z is a cationic organic fragment and X is an anion.

9. A method according to claim 8 wherein at least one group Z is a fragment of formula
[Y(R¹)₄]⁺
where Y is N, P or As, and R¹ groups are the same or different and are selected from H, hydrocarbon groups or heteroatom-substituted hydrocarbon groups.

10. A method according to claim 9 wherein the R¹ groups comprise one or more of:
linear, branched or cyclic alkyl groups with 1 to 50 carbon atoms (preferably selected from methyl, ethyl, n-propyl, i-propyl, butyl, pentyl, hexyl, octyl, decyl, cyclopentyl, cyclohexyl); aryl or substituted aryl groups (preferably selected from phenyl, ortho-tolyl, meta-tolyl, para-tolyl, ethylphenyl, isopropylphenyl, t-butylphenyl, 2,6-dimethylphenyl, 2,4-dimethylphenyl, 3,5-dimethylphenyl, 2,6-diisopropylphenyl, 2,4,6-trimethylphenyl, 2,4,6-triisopropylphenyl, naphthyl, benzyl); heteroatom-substituted hydrocarbon groups (preferably selected from CF₃, CF₂CF₃, CH₂OMe, CH₂NMe₂, CH₂CH₂NH₂, CH₂CH₂N(R⁵)₂, CH₂CH₂P(R³)₂, CH₂CH₂CH₂P(R⁵)₂, fluorophenyl, perfluorophenyl, chlorophenyl, bromophenyl, C₆H₄(CF₃), C₆H₃(CF₃)₂, C₆H₄(OMe), C₆H₃(OMe)₂, C₆H₄(N(R⁵)₂), C₆H₄(P(R⁵)₂), where R⁵ is selected from H, hydrocarbon groups or heteroatom-substituted hydrocarbon groups); or two or more R¹ groups may be linked so as to form a cyclic structure.

11. A method according to claim 8 wherein Z is a heterocycle of formula (I): 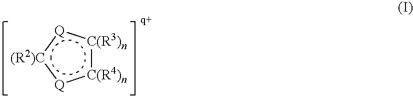
wherein the broken line within the ring structure indicates that one or more pairs of adjacent ring atoms are multiply bonded;
the Q groups may be the same or different and are O, S, N(R¹), P(R¹) or C(R¹)₂ where R¹ is as defined in claim 8 or 9;
the groups R²—R⁴ may be the same or different and have the same definition as R¹ in claim 9 or 10;
the values of n may be the same or different and are each 1 or 2; and
any of the groups R¹—R⁴ may be linked to form cyclic structures.

12. A method according to any of claims 8 to 11 in which the or each X is an anion selected from F, Cl, Br, I, acetate, triflate, tosylate, BF₄, AlCl₄, PF₆, ClO₄BPh₄, B(C₆F₅)₄, B[3,5-(CF₃)₂C₆H₄]₄, and Al(OC₄F₉)₄.

13. The method of any preceding claim wherein the acid is selected from HF, HCl, HBr, HI, tosylic acid, fluorinated organic acids, HBF₄, acetic acid, solid-state acids, and heteropolyacids.

14. The method of claim 13 in which the acid is HBr or HI.

15. The method of any preceding claim wherein component (ii) is said compound consisting of said conjugate base and radical, produced by pre-reacting the acid with some of the alcohol substrate before addition of the other catalyst components.

16. The method of any preceding claim in which the catalyst composition includes said ligand (iv)

17. The method of claim 16 in which said ligand is a monodentate or polydentate C-, N-, P-, As-, O- or S-donor ligand.

18. The method of claim 16 or 17 in which said ligand (iv) is based on C-donors selected from CO or carbenes, N-donors selected from amine, pyridine and bipyridine; O-donors selected from acetate and acetyl acetonate; and/or P-donors selected from phosphines.

19. The method of any preceding claim wherein, in the catalyst composition, the ratio of the moles of the Group VIII transition metal in component (1) to the organic salt (2) is in the range 1:1 to 1:10⁸; the ratio of the moles of the Group VIII transition metal in component (1) to the acid (3) is in the range 1:1 to 1:10⁸; and the ratio of the moles of the Group VIII transition metal in component (1) to the ligand (4) is in the range 0.01:1 to 1:100.

20. The method of any one of the preceding claims, wherein the alcohol is selected from ethanol, propanol, butanol or hexanol.

21. The method of any one of the preceding claims, wherein the product of the dehydration reaction is predominantly alkene.

22. The method of any one of claims 1 to 20, wherein the product of the dehydration reaction is predominantly ether.

23. The method of any preceding claim wherein the reaction temperature is in the range from 50° C. to 200° C.

24. The method of claim 23 wherein the reaction temperature is from 70° C. to 150° C.

25. The method according to any preceding claim wherein reaction occurs in the presence of said catalyst composition and a second catalyst capable of converting the product produced in the dehydration reaction into a further product preferably under the conditions employed for the dehydration reaction.

26. The method of claim 25 in which said second catalyst is selected from catalysts for alkene dimerisation, trimerisation, oligomerisation or polymerisation.

27. The method of claim 25 or 26 wherein the second catalyst comprises a source of a Group VIII metal.
